(19) United States
(12) Patent Application Publication
(10) Pub. No.: US 2017/0128288 A1
(43) Pub. Date: May 11, 2017

(54) LIGHT WEIGHT MULTI USE COLLAPSIBLE STRETCHER

(71) Applicant: TELESTRETCH LTD., KAHAL (IL)

(72) Inventors: Dan SION, KAHAL (IL); YINBAL Alazar C., ENCINO, CA (US)

(73) Assignee: TELESTRETCH LTD., KAHAL (IL)

(21) Appl. No.: 15/379,504

(22) Filed: Dec. 15, 2016

Related U.S. Application Data
(63) Continuation-in-part of application No. 14/409,113, filed on Dec. 18, 2014, now abandoned.
(60) Provisional application No. 61/662,395, filed on Jun. 21, 2012.

(51) Int. Cl. A61G 1/013; A61G 1/048; A61F 17/00; A61G 1/044

(52) U.S. Cl. CPC A61G 1/013; A61G 1/044; A61G 1/048; A61F 17/00

(57) ABSTRACT

A lightweight multi-use collapsible stretcher for carrying a load comprising: two opposing collapsible longitudinal frames; at least one bedding member disposed between said longitudinal frames; and at least one interconnecting bar disposed between said two opposing longitudinal frames and hingedly connected to at least two longitudinal frames. The interconnecting bar comprises first and second sections, which have a position attached to said frame plates when said stretcher is in said folded position and a position transversely deployed towards each other when said stretcher is in said unfolded position. The interconnecting bar has interconnecting means at at least one free terminal thereof; said two opposing collapsible longitudinal frames are interconnectable via interconnecting bar and said interconnecting means in said unfolded position.

### CROSS-REFERENCE TO RELATED APPLICATIONS

This Application is a Continuation-In-Part Application of U.S. patent application Ser. No. 14/409,113, filed on Dec. 18, 2014, which claims priority from PCT Application No. PCT/IL2013/050523, filed Jun. 18, 2013, and U.S. Provisional Patent Application No. 61/662,395, filed on Jun. 21, 2012. All of these applications are hereby incorporated by reference in their entirety.

### BACKGROUND OF THE INVENTION

Stretchers are emergency evacuation devices used to carry casualties or an incapacitated people evacuate and convey them from one place to another. A stretcher is usually carried by two people or more, at the head and at the feet of the incapacitated person. The casualty is placed on the stretcher, and can then be carried or wheeled away. Stretchers are indicated if a person is either unable to walk or walking by him/herself may compromise his/her health, or if other requirements such as a wheelchair, or similar device cannot be used.

It has been well established that a stretcher needs to be lightweight, strong enough, durable and foldable, as well as very quickly and easily erectable. It is a further requirement that stretcher would be transformable and modified for multi uses needed such as: ladder, bridge, electronic device carrier, airlift, wheeled carriage and other similar devices. To meet this need, stretchers having multiple fold lines have been developed. Such stretchers are readily transported in its folded state, and are sufficiently compact to allow a medic or any rescue crewmember to carry a folded stretcher by hand or on the back.

Zalman U.S. Pat. No. 4,685,161 discloses a foldable framework for objects such as: stretchers, beds, bridges and the like. The framework comprises first and second carrier rod assemblies linked to each other by foldable connecting means, each such assembly comprising four rods linked by three joints, a central joint and two peripheral joints so designed that the extreme rods of the two carrier rod assemblies swing in different planes.

Guo CN2305977 discloses an M-shaped four-section and three-fold portable stretcher for the field of the rescue and the life saving. The weight of the stretcher is about 4 kilograms and is suitable for one person walking and carrying, and can also be arranged in a micro-miniature motor vehicle to carry.

Chaojun CN201572259U discloses a dual-use portable folding stretcher mainly comprises two supporting rods and a supporting surface, wherein the two supporting rods pass through the two long sides of the rectangular supporting surface and adopt the structures formed in the manner that two sections of insertion pipes are connected and fixed in a mutually-inserted manner; a layer of air-filled mattress are arranged below the supporting surface; rolling wheels are installed at the corners of the bottom surface of the air-filled mattress; and the surface corners of the supporting surface are respectively connected with bandages. The dual-use portable folding stretcher has the advantages of reasonable structure, convenience, practicability, flexible operation and the like. Sion and Yinbal WO2010131247 disclosed in a former document a foldable stretcher for transporting an object, comprising: a plurality of substantially plane members pivotally connected with each other in series by means of hinges there between; handles pivotally connected to outermost of the plane members; and, at least two pluralities of props. The stretcher further comprises at least two pairs of sufficiently stiff wires. Each pair of the wires arranged along a corresponding long side of the stretcher; each the wire is mechanically connected to the stretcher at least two connection points.

An improved stretcher of a more convenient structure is a long felt unmet need.

### SUMMARY OF THE INVENTION

It is one object of the present invention to provide a lightweight multi-use collapsible stretcher [100] for carrying a load. The collapsible stretcher [100] comprising: (a) two opposing collapsible longitudinal frames [110], each collapsible longitudinal frame comprising at least four frame plates interconnected by hinges [140] disposed therebetween and providing reversible operation of unfolding and folding of the stretcher into an unfolded and folded position; (b) at least one bedding member [130], disposed between the longitudinal frames; (c) at least one interconnecting bar [210], disposed between the two opposing longitudinal frames and hingedly connected to at least two longitudinal frames [110].

It is a core purpose of the invention to provide the interconnecting bar comprising first and second sections. The first and second sections [210a] and [210b] have a position attached to the frame plates [120] when the stretcher [100] is in the folded position and a position transversely deployed towards each other when the stretcher [100] is in the unfolded position. The interconnecting bar [210] has interconnecting means at at least one free terminal thereof. The two opposing collapsible longitudinal frames are interconnectable via interconnecting bar [210] and the interconnecting means in the unfolded position.

Another object of the invention is to disclose at least one interconnecting bar [210] hingedly connected to one of the collapsible longitudinal frames. The first and second sections [210a] and [210b] are hinged therebetween. A free terminal of the at least one interconnecting bar [210] is provided with a T-slider insertable into a T-shaped slot located at an opposing longitudinal frame [110].

Another object of the invention is to disclose the interconnecting means comprising cooperatively disposed a pin and a coupling arresting member having a shoulder at the free terminal of the first section and a first slot, and a spring-loaded lock. The slot is configured for receiving the pin. The shoulder is configured for catching the spring-loaded lock when the spring is manually compressed and anchor the spring-loaded lock thereon.

A further object of the invention is to disclose the interconnecting means comprises a reciprocatively movable latch bar at the free terminal of the first section and a second slot configured for receiving the latch bar at the free terminal of the second section when the first and second sections are deployed.

A further object of the invention is to disclose a free terminal of the first section having a slot configured for receiving a free terminal of the second section. The first and second section are arrangeable angularly to each other and rectifiable around a pin disposed within the slot within the free terminal of the first section. The free terminal of the second section has a recess for receiving the pin and a second spring-loaded lock clamping the free terminal of the first section on the free terminal of the second section.

A further object of the invention is to disclose the first and second sections mechanically fixatable when the first and second sections are deployed.

A further object of the invention is to disclose the articulated linkage comprising two levers hinged therebeween.

A further object of the invention is to disclose the external ends of the terminal frame plates comprising grip-handles.

A further object of the invention is to disclose the grip-handles covered with a material selected from a group consisting of: rubber, silicon, foam, plastic, composite material and any combination thereof.

A further object of the invention is to disclose the grip-handles which are foldable or telescopically shortened.

A further object of the invention is to disclose at least one of the plurality of hinges which is a dual axis rotating hinge.

A further object of the invention is to disclose at least one of the hinges which is a double action hinge.

A further object of the invention is to disclose the bedding member comprising of at least one fabric strap or mesh.

A further object of the invention is to disclose the bedding member configured for reversible removal from the two opposing longitudinal frames.

A further object of the invention is to disclose the bedding member comprising at least four rigid platform members.

A further object of the invention is to disclose the platform members connected to the longitudinal frame by flexible connectors or by the hinges configured to allow the longitudinal frame to be rotated towards and away from the platform members.

A further object of the invention is to disclose the number of the platform members same as the number of the frame plates.

A further object of the invention is to disclose the platform members made of a material selected from the group consisting of: metal, polymeric material, composite material, wood and any combination thereof.

A further object of the invention is to disclose the platform members having a shape selected from a group consisting of a grate-like, a honeycomb-like, a perforated shape, a pole frame and any combination thereof.

A further object of the invention is to disclose the stretcher further comprising back splint or straps, for securing the load to the stretcher

A further object of the invention is to disclose the load selected from a group consisting of: a patient, an individual of limited capability, a disabled person, an injured person, a victim of an emergency situation, an animal, an unanimated object and any combination thereof.

A further object of the invention is to disclose the frame plates made of a material selected from a group consisting of: metal, polymeric material, composite material, wood and any combination thereof;

A further object of the invention is to disclose the stretcher comprising connectors for attaching at least two of the stretchers in parallel- or in sequential-series configuration.

A further object of the invention is to disclose the stretcher further comprising connectors for attaching electric and electronic equipment. The stretcher further comprises connectors for attaching carrying extension cables or ropes.

A further object of the invention is to disclose the stretcher further comprising fixation elements configured to fix the load to the stretcher by means of tying.

A further object of the invention is to disclose the stretcher made of material at least partially transparent to X-rays and electro-magnetic fields.

A further object of the invention is to disclose the stretcher comprising a live saving kit.

A further object of the invention is to disclose the live saving kit comprising an item selected from a group consisting of: tourniquet means, needles, sutures, bandages, vital monitoring devices, intravenous therapy means, telescopic rod for fixating intravenous means, a defibrillator, a tracheostomy means, a beg-valve resuscitator, a pneumatic resuscitator, a pneumatic/electrically powered portable lung ventilator, a transmitter for transmitting a patient's vitals and status endotracheal-tube, tracheostomy means and any combination thereof.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 is a perspective view of a light multi-use stretcher in an unfolded position;

FIGS. 2a to 2c are perspective views of a light multi-use stretcher in a partially and fully folded position;

FIG. 3 is a perspective view of a light multi-use stretcher with four rigid platform members;

FIGS. 4a to 4f are perspective views of a horizontal clamping mechanism coupling separable sections of an interconnecting bar;

FIGS. 5a to 5c are perspective views of a latching mechanism coupling separable sections of an interconnecting bar;

FIGS. 6a to 6e are perspective views of a light multi-use stretcher stepwise illustrating deploying interconnecting bars with a vertical clamping mechanism;

FIGS. 7a to 7c are views of an alternative clamping mechanism coupling separable sections of an interconnecting bar;

FIGS. 8a to 8c are perspective views of a T-shaped interconnecting means;

FIGS. 9a and 9b are perspective views of a light multi-use stretcher provided with one and three interconnecting bars, respectively.

### DETAILED DESCRIPTION OF THE INVENTION

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a novel and useful stretcher device and methods.

The present invention is a light multi-use collapsible stretcher suitable for carrying wounded humans, animals or equipment including electrical equipment. Also the new light multi-use stretcher can be used as a bed, bridge or a ladder.

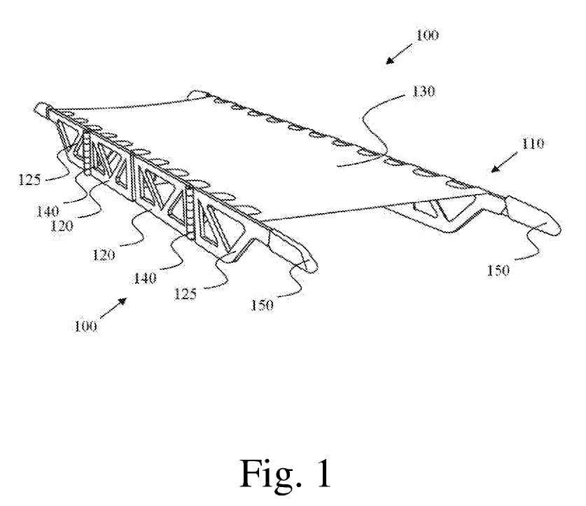

Reference is now made to FIG. 1 presenting lightweight multi-use collapsible stretcher [100 ]. Aforesaid stretcher [100] comprises: a) two opposing longitudinal frames [110], each comprising at least four frame plates [120]; b) at least one bedding member [130], disposed between longitudinal frames [110]; c) plurality of hinges [140]; and d) at least one foldable and reversibly fastenable interconnecting bar [210], disposed between the two opposing longitudinal frames [110] and pivotally connected to two opposing longitudinal frames [110] by hinges [140].

Frame plates [120] are pivotally connected to each other by hinges [140] in a series configuration. Hinges [140] disposed between frame plates [120] in alternate rotation directions, such that frame plates [120] and bedding member [130] configured for a reversible operation of unfolding and folding of stretcher [100] into an unfolded and folded position.

In one embodiment of the present invention the at least one interconnecting bar [210] is configured to be detached from one of the opposing longitudinal frames [110] and to be attached to opposing longitudinal frame [110], for the purpose of folding stretcher [100]. In another embodiment the at least one interconnecting bar [210] is comprises two reversibly separable sections [210]; each of sections [210] configured to be separated from the other and to be attached to corresponding longitudinal frame [110] (the one it is hinged to), for the purpose of folding stretcher [100].

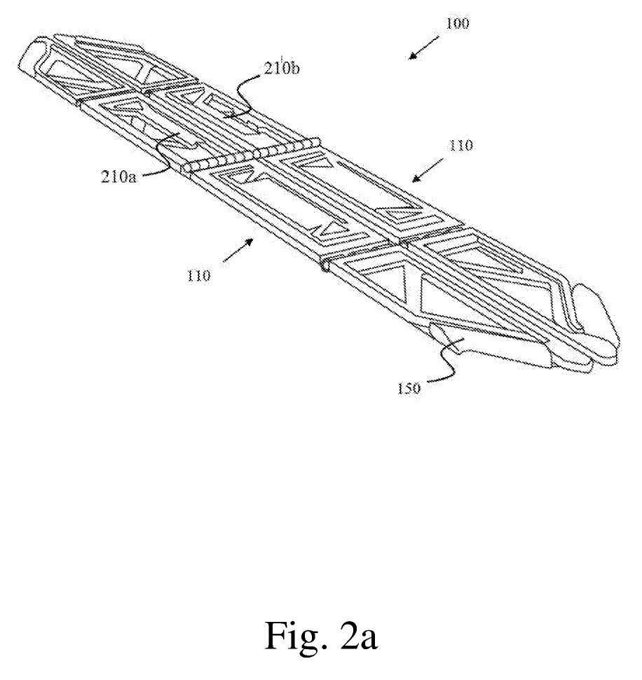

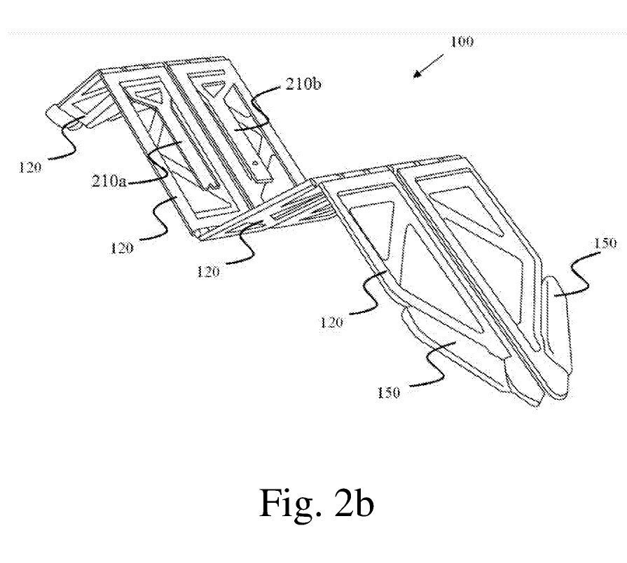

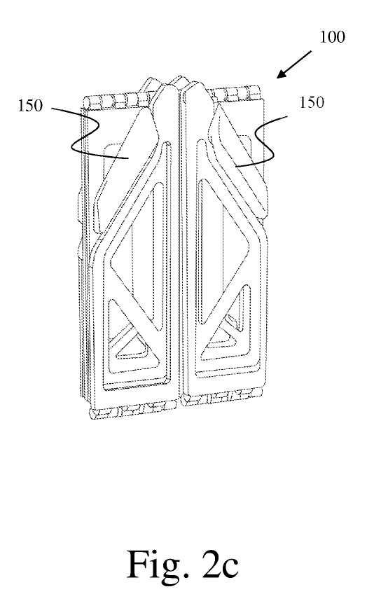

In another preferred embodiment the two longitudinal frames [110] are configured as a beam with a rectangular or an I-shape cross-section, for bearing the loads force vectors. In one embodiment, bedding member [130] is a flexible fabric bedding, as demonstrated in FIGS. 1 and 2, allowing the folding and unfolding of stretcher [100]. Two terminal frame plates [125] of longitudinal frames [110] are provided with grip-handles [150] at the ends thereof.

Reference is now made to FIGS. 2a to 2c, presenting the folding procedure of stretcher in a stage-by-stage manner. It should be emphasized that frames [110] in FIG. 2a are turned by 270° in comparison with unfolded position in FIG. 1. Detaching of bedding 130 from frames [110] is also in the scope of the present invention. FIGS. 2b and 2c illustrate termination of the folding procedure.

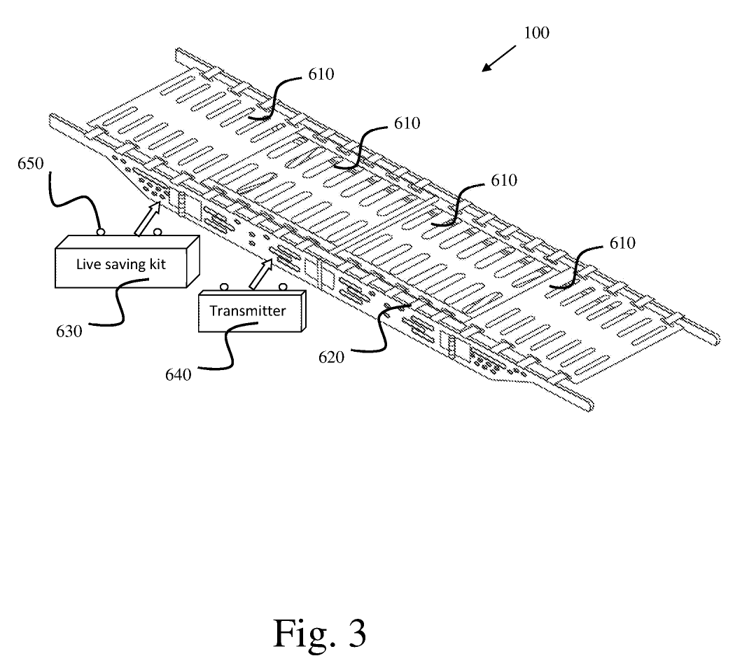

Reference is now made to FIG. 3, presenting bedding member [130] constructed of four rigid platform members [610]. Platform members [610] are attached to the four frame plates [120] by means of plurality of flexible connectors [620] or hinges [140] (not shown) which allow two opposing longitudinal frames [110] to be rotated towards and away from platform members [610].

FIG. 3 further demonstrates another embodiment of the invention where collapsible stretcher [100] further comprises live saving kit [630], connected by connectors [650] to the body of stretcher [100]. Live saving kit [630] can include items such as: tourniquet means, needles, sutures, bandages, vital monitoring devices, intravenous therapy means, telescopic rod for fixating intravenous means, endotracheal-tube, tracheostomy means and any combination thereof. In another embodiment, collapsible stretcher [100] may further include transmitter [640] for transmitting the patient's vitals and status to an acting physician and/or to a receiving medical center.

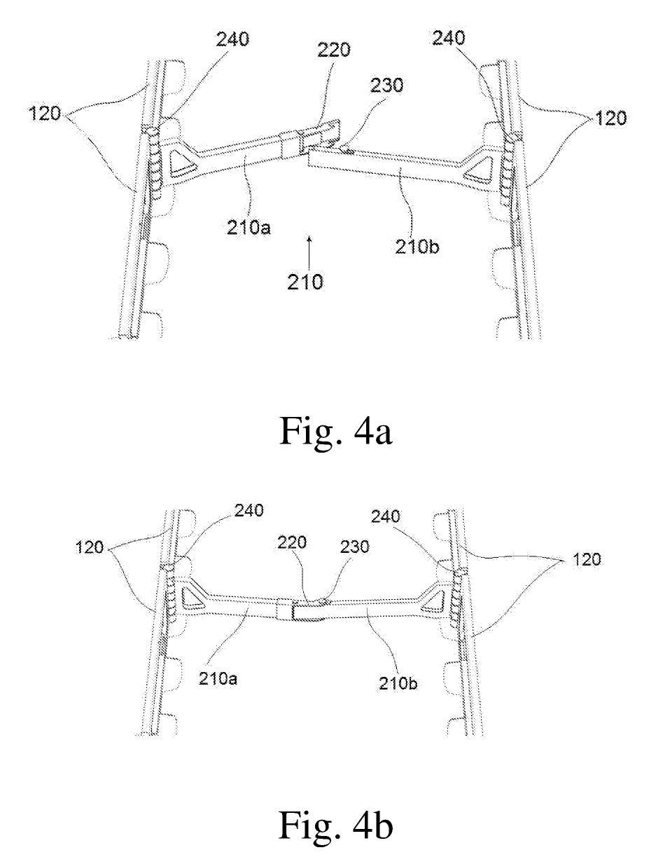

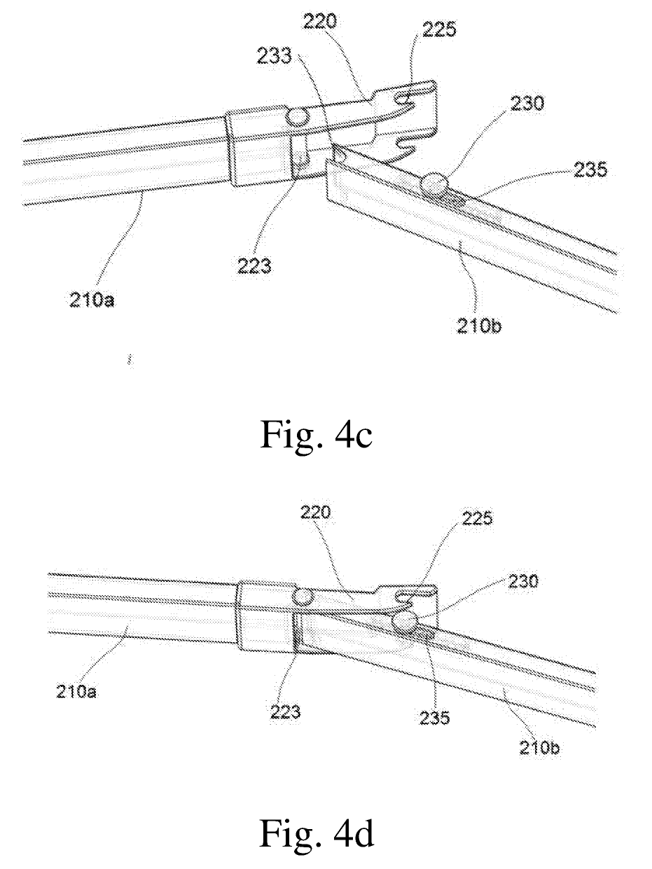

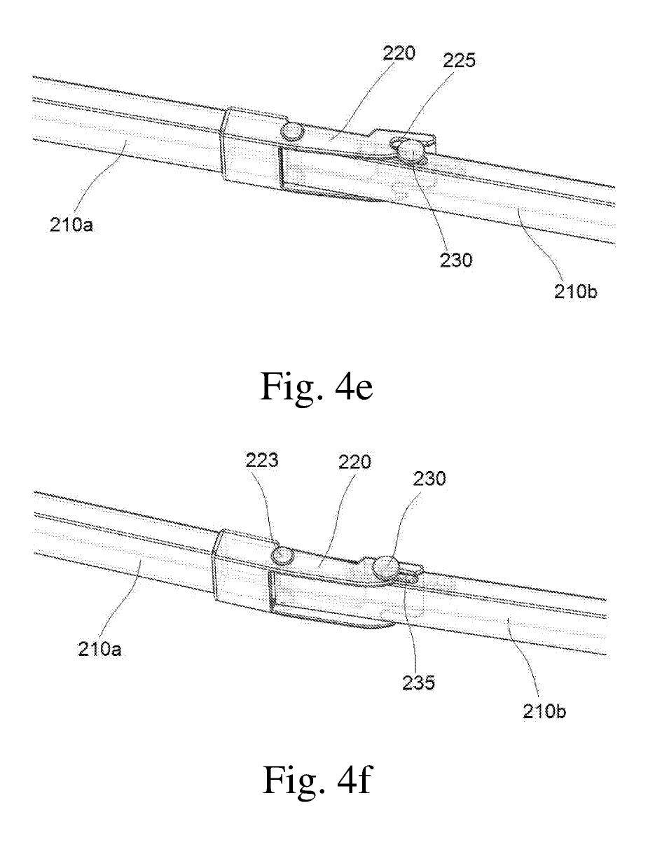

Reference is now made to FIGS. 4a to 4f, presenting the first embodiment of the interconnecting means. First and second sections 210a and 210b of the interconnecting bar are shown in detached and coupled positions. Pin 223 and a coupling arresting member 220 having a shoulder 225 at the free terminal of first section 210a and optional slot 233 and spring-loaded lock 230. First slot 233 is configured for receiving pin 233. Shoulder 225 is configured for catching spring-loaded lock 230 when spring 235 is manually compressed and anchor spring-loaded lock 230 thereon. Similar clamping mechanism can be used in a vertical way of coupling separating sections instead of horizontal coupling.

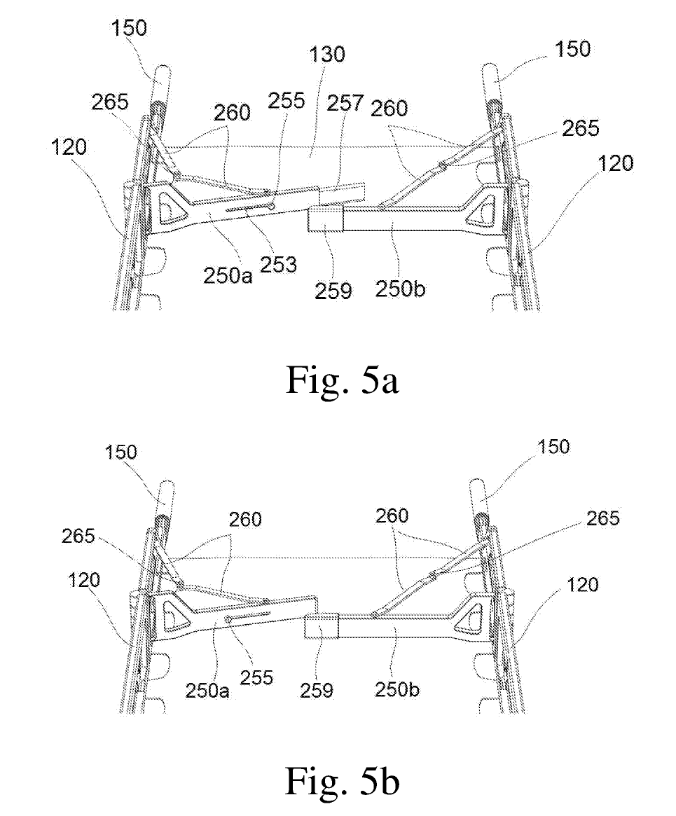

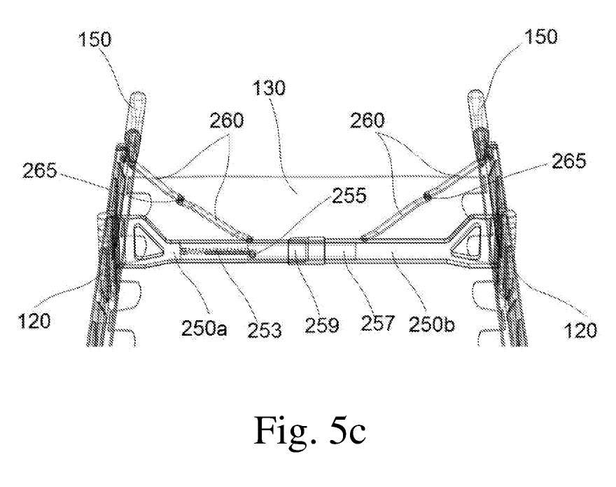

Reference is now made to FIGS. 5a to 5c presenting the second embodiment of the interconnecting means. Reciprocatively movable latch bar 257 at the free terminal of first section 250a and second slot 259 configured for receiving latch bar 257 at free terminal of said second section 250b when first and second sections 210a and 210b are deployed. Latch bar 257 is spring-loaded by spring 253 and manually manipulated by handle 255.

First and second sections 210a and 210b are mechanically fixable by an articulated linkage when they are deployed. The aforesaid articulated linkage comprises two levers 260 interconnected by hinge 265.

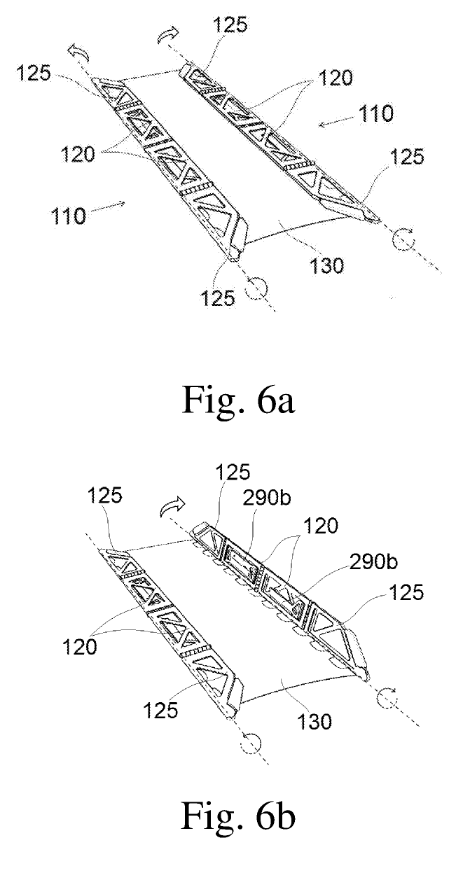

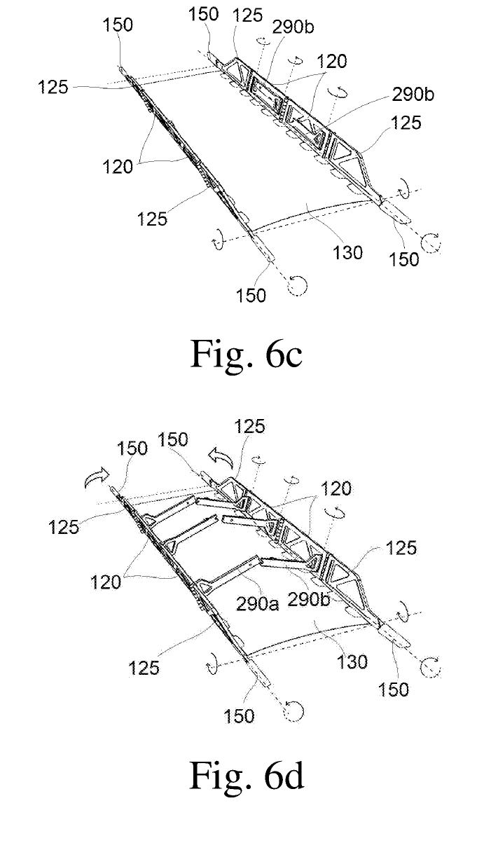

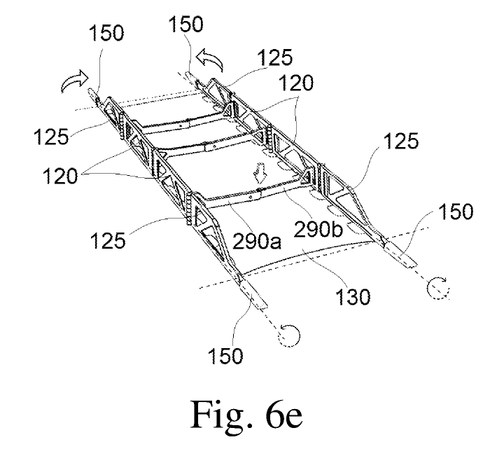

Reference is now made to FIGS. 6a to 6e showing stepwise deploying first and second sections 290a and 290b of interconnecting bars. Specifically, FIG. 6a a position of side longitudinal frames 110 rotated by 270° relative to the initial position. FIG. 6b depicts up-righting of side longitudinal frames 110. Then, FIGS. 6c and 6d show deploying first and second sections 290a and 290b and arranging them angularly to each other. Grip-handles 150 are also put into the operation position. FIG. 6e presents the stretcher with locked first and second sections 290a and 290b.

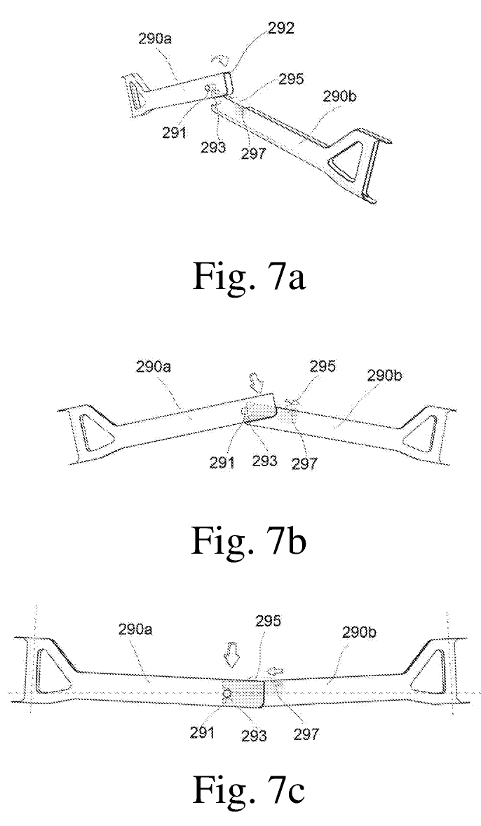

Reference is now made FIGS. 7a to 7c presenting stepwise locking first and second sections 290a and 290b of the interconnecting bar. a free terminal of first section 290a has a slot 292 configured for receiving a free terminal of second section 290b. First and second sections 290a and 290b are arrangeable angularly to each other and rectifiable around a pin 291 disposed within said slot 292 within the free terminal of first section 290a. The free terminal of second section 290b has recess 293 for receiving pin 291 and lock 295 loaded by spring 297 and clamping the free terminal of first section 290a on the free terminal of second section 290b.

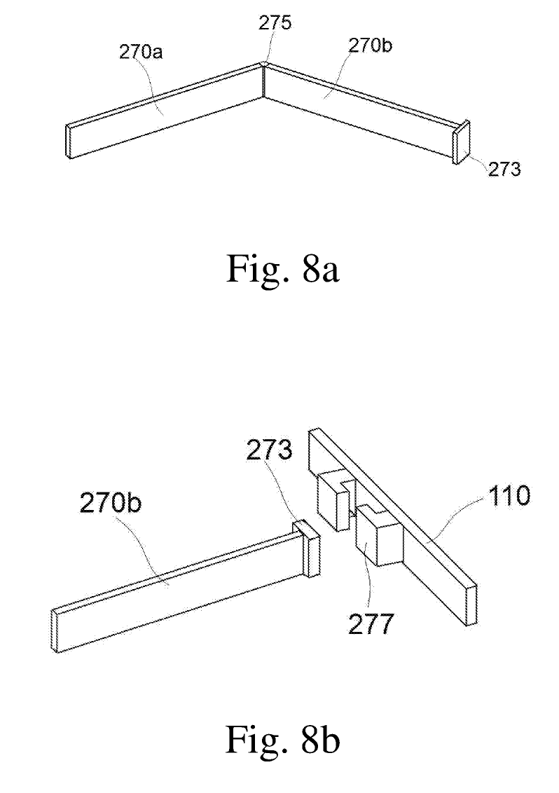

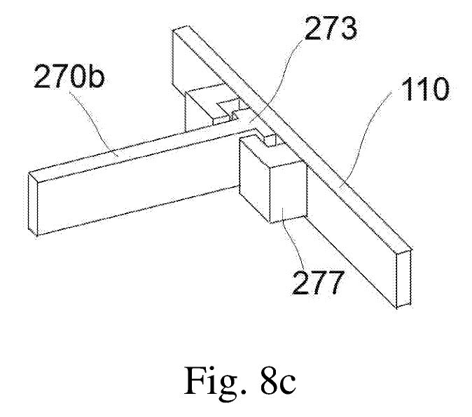

Reference is now made FIGS. 8a to 8c presenting an alternative embodiment of the present invention. First and second sections 270a and 270b are hinged therebetween. A free terminal of first section 270a is hingedly connected to opposing collapsible longitudinal frame 110 (not shown). Second section 270b is provided with T-shaped slider 273 insertable into T-shaped slot 277 located on opposing collapsible longitudinal frame 110.

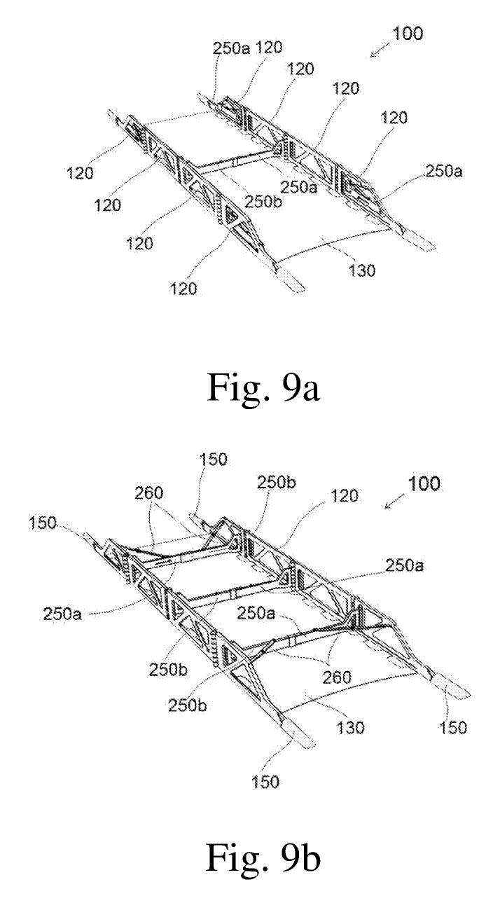

Reference is now made to FIGS. 9a and 9b presenting general perspective views of stretcher 100 from below. In FIG. 9a, one pair of sections 250a and 250b are interconnected while and two other are detached. In FIG. 9b, all pairs of sections 250a and 250b are interconnected.

It will be appreciated by a person skilled in the art that the present invention is not limited by what has been particularly shown and described hereinabove. Rather the scope of the present invention includes both combinations and sub-combinations of the features described hereinabove as well as modifications and variations thereof which would occur to a person of skill in the art upon reading the foregoing description and which are not in the prior art.

1. A lightweight multi-use collapsible stretcher [100] for carrying a load, comprising:
a) two opposing collapsible longitudinal frames [110], each collapsible longitudinal frame comprising at least two frame plates interconnected by hinges [140] disposed therebetween and providing reversible operation of unfolding and folding of said stretcher into an unfolded and folded position;
b) at least one bedding member [130], disposed between said longitudinal frames;
c) at least one interconnecting bar [210], disposed between said two opposing longitudinal frames and hingedly connected to at least two longitudinal frames[110];
wherein said interconnecting bar comprises first and second sections; said first and second sections [210a/250a/270a/290a] and [210b/250b/270b/290b] have a position attached to said frame plates [120] when said stretcher [100] is in said folded position and a position transversely deployed towards each other when said stretcher [100] is in said unfolded position; said interconnecting bar [210] has interconnecting means at at least one free terminal thereof; said two opposing collapsible longitudinal frames are interconnectable via interconnecting bar [210] and said interconnecting means in said unfolded position.

2. The collapsible stretcher according to claim 1, wherein at least one interconnecting bar is hingedly connected to one of said collapsible longitudinal frames; said first and second sections [270a] and [270b] are hinged therebetween; a free terminal of said at least one interconnecting bar [210] is provided with a T-slider insertable into a T-shaped slot located at an opposing longitudinal frames [110].

3. The collapsible stretcher according to claim 1, wherein said interconnecting means comprises cooperatively disposed a pin and a coupling arresting member having a shoulder at said free terminal of said first section and a first slot, and a first spring-loaded lock; said slot is configured for receiving said pin; said shoulder is configured for catching said spring-loaded lock when said spring is manually compressed and anchor said first spring-loaded lock thereon.

4. The collapsible stretcher according to claim 1, wherein said interconnecting means comprises a reciprocatively movable latch bar at said free terminal of said first section and a second slot configured for receiving said latch bar at said free terminal of said second section when said first and second sections are deployed.

5. The collapsible stretcher according to claim 1, wherein a free terminal of said first section has a slot configured for receiving a free terminal of said second section; said first and second section are arrangeable angularly to each other and rectifiable around a pin disposed within said slot within said free terminal of said first section; said free terminal of said second section has a recess for receiving said pin and a second spring-loaded lock clamping said free terminal of said first section on said free terminal of said second section.

6. The collapsible stretcher according to claim 1, wherein said first and second sections are mechanically fixatable when said first and second sections are deployed.

7. The collapsible stretcher according to claim 1, wherein said articulated linkage comprises two levers hinged therebeween.

8. The collapsible stretcher according to claim 1, wherein said external ends of the external said frame plates comprise grip-handles.

9. The collapsible stretcher according to claim 7, wherein said grip-handles are covered with a material selected from a group consisting of: rubber, silicon, foam, plastic, composite material and any combination thereof.

10. The collapsible stretcher according to claim 7, wherein said grip-handles are foldable or telescopically shortened.

11. The collapsible stretcher according to claim 1, wherein at least one of the following is true:
a. at least one of said plurality of hinges is a dual axis rotating hinge;
b. at least one of said hinges is a double action hinge;
c. said bedding member comprises of at least one fabric strap or mesh;
d. said bedding member is configured for reversible removal from said two opposing longitudinal frames; and
e. said bedding member comprises at least four rigid platform members.

12. The collapsible stretcher according to claim 11, wherein said platform members are connected to said longitudinal frame by flexible connectors or by said hinges, configured to allow said longitudinal frame to be rotated towards and away from said platform members.

13. The collapsible stretcher according to claim 11, wherein the number of the platform members is same as the number of the frame plates.

14. The collapsible stretcher according to claim 11, wherein said platform members are made of a material selected from the group consisting of: metal, polymeric material, composite material, wood and any combination thereof.

15. The collapsible stretcher according to claim 11, wherein said platform members have a shape selected from a group consisting of a grate-like, a honeycomb-like, a perforated shape, a pole frame and any combination thereof.

16. The collapsible stretcher according to claim 1, wherein at least one of the following is true:
a. said stretcher further comprises back splint or straps, for securing said load to said stretcher;
b. said load is selected from a group consisting of: a patient, an individual of limited capability, an disabled person, an injured person, a victim of an emergency situation, an animal, an unanimated object and any combination thereof;
c. said frame plates are made of a material selected from a group consisting of: metal, polymeric material, composite material, wood and any combination thereof;
d. said stretcher comprises connectors for attaching at least two of said stretchers in parallel- or in sequential-series configuration;
e. said stretcher further comprises connectors for attaching electric and electronic equipment said stretcher further comprises connectors for attaching carrying extension cables or ropes.

17. The collapsible stretcher according to claim 1, wherein said stretcher further comprises fixation elements configured to fix said load to said stretcher by means of tying.

18. The collapsible stretcher according to claim 1, wherein said stretcher is made of material at least partially transparent to X-rays and electro-magnetic fields.

19. The collapsible stretcher according to claim 1, wherein said stretcher further comprises a life saving kit.

20. The collapsible stretcher according to claim 19, wherein said live saving kit comprises an item selected from a group consisting of: tourniquet means, needles, sutures, bandages, vital monitoring devices, an intravenous therapy means, a telescopic rod for fixating intravenous means, a defibrillator, an endotracheal-tube, tracheostomy means, a beg-valve resuscitator, a pneumatic resuscitator, a pneumatic/electrically powered portable lung ventilator, a transmitter for transmitting a patient's vitals and status and any combination thereof.
